(19) United States
(12) Patent Application Publication
(10) Pub. No.: US 2026/0294757 A1
(43) Pub. Date: Oct. 1, 2026

(54) ANTIPERSPIRANT COMPOSITION

(71) Applicant: Conopco, Inc., d/b/a UNILEVER, Hoboken, NJ (US)

(72) Inventors: Somnath DAS, Bangalore (IN); Praveen NYALAM, Karimnagar, Telangana (IN); Satyajit SAMADDER, Bangalore (IN)

(21) Appl. No.: 19/483,933

(22) PCT Filed: Jun. 3, 2024
(86) PCT No.: PCT/EP2024/065185

§ 371 Date: Nov. 12, 2025

(30) Foreign Application Priority Data
Jun. 7, 2023 (EP) 23177877.0

(51) Int. Cl. A61K 8/28; A61K 8/26; A61K 8/41; A61K 8/49; A61K 8/60; A61K 8/891; A61Q 15/00

(52) U.S. Cl. CPC A61K 8/28; A61K 8/26; A61K 8/416; A61K 8/4973; A61K 8/602; A61K 8/891; A61Q 15/00

(57) ABSTRACT

The present invention is in the field of providing an antiperspirant composition for minimizing stains, particularly the yellowish discoloration on fabrics. The present invention is directed to an antiperspirant composition comprising a metal based antiperspirant active, non-ionic surfactant having an HLB value less than 12 and cationic surfactant.

### FIELD OF THE INVENTION

The present invention relates to an antiperspirant composition. The invention particularly relates to an antiperspirant composition for reducing stains on fabrics.

### BACKGROUND OF THE INVENTION

Many consumers complain about undesirable stains in the underarm area in clothing with repeated use of antiperspirants. These are frequently yellowish stains which may also tend to become incrusted. These stains are produced due to a complex interaction between antiperspirant composition, skin fat, sweat and are often difficult to remove with conventional washing methods.

Certain astringent metal salts such as aluminium or zirconium salts e.g., aluminium chlorohydrates or sesquichlorohydrates are widely used as antiperspirant active in antiperspirant composition. These actives have the effect of reducing perspiration, particularly when applied onto the underarm regions of the human body viz, the axilla. However, a drawback of the presence of the metal based antiperspirant active is that it may cause stains.

These stains are particularly formed in the underarm region. These stains are formed when the fabric comes in contact with the antiperspirant composition while wearing and may remain on the fabric after washing. The stains become more intensive after several such wear and wash cycles and appear as yellow discoloration on fabrics. The stains of particular concern are the yellow discoloration which are produced when the antiperspirant composition reaches the textile during sweating along with the body secretions, particularly in the armpit region. In combination with the sweat the antiperspirant actives form a yellow stain. Further, in a normal machine-washing cycle this yellow stain is accentuated by washing, and the heat (electric dryer, ironing or pressing) can further accentuate the intensity of the coloring. Moreover, the yellowing can be transferred to other areas of the clothing during laundering of the clothing. The combination of materials that contribute to yellowing can be redeposited to other areas of the clothing during laundering. The yellowing is more noticeable on white or light-colored clothing, while particulate deposits and stiffness are prevalent on all types of fabrics.

The problem of yellow discoloration is further aggravated when the antiperspirant composition includes unsaturated oils. Increasingly, current antiperspirant compositions also include natural oils for delivering skin care benefits. The natural oil includes unsaturated oils such as sunflower seed oil.

US2014/093464 A1 (Urban Michael et. al. 2014) discloses an antiperspirant composition including metal based antiperspirant active, nonionic surfactant and cationic surfactant.

It is an object of the present invention to provide an antiperspirant composition for minimizing the stains on clothing caused by the interaction of body oils with antiperspirant composition having a metal based antiperspirant active or those caused by the interaction of the antiperspirant active with the oils present in the composition.

It is another object of the present invention to provide an antiperspirant composition for minimizing the stains on clothing caused by the interaction of body oils with antiperspirant composition having a metal based antiperspirant active and where the antiperspirant composition is anhydrous.

### SUMMARY OF THE INVENTION

The present inventors have surprisingly found that when specific nonionic surfactant having a HLB value less than 12 and specific levels of cationic surfactant is included in an antiperspirant composition comprising a metal based antiperspirant active, then the stain on clothing caused by the interaction of body oils with antiperspirant composition having a metal based antiperspirant active or those caused by the interaction of the antiperspirant active with the oils present in the composition is easily removed when the fabric is subsequently washed. The inventive anhydrous aerosol antiperspirant composition provides for reducing or eliminating the yellowing of clothing while still maintaining the effectiveness of the antiperspirant composition. The composition also provides for improving washability of stains and reducing the stiffness of the fabrics resulting from the deposition of the antiperspirant composition on fabrics. It was also found that the composition according to the present invention provides for reducing particulate build up on the fabrics on repeated wash wear, thus reducing the white marks on dark coloured/black fabrics.

According to the first aspect of the present invention disclosed is an anhydrous aerosol antiperspirant composition comprising:
(i) a metal based antiperspirant active;
(ii) 0.5 wt. % to 6 wt. % non-ionic surfactant having an HLB value less than 12; and,
(iii) 0.1 wt. % to 4 wt. % cationic surfactant.
wherein the nonionic surfactant is selected from the group consisting of polyoxyethylene sorbitan alkyl esters (sold as Tween surfactants), fatty alcohol ethoxylates (sold as Brij surfactants), sorbitan monoester (sold as Span surfactants), alkyl polyglucosides, and C₈-C₁₆ fatty alcohol glycoside.

According to a second aspect of the present invention disclosed is a use of a non-ionic surfactant having an HLB value less than 12; and a cationic surfactant in an antiperspirant composition comprising a metal based antiperspirant active for minimizing the stains on the fabrics caused by antiperspirant composition.

According to another aspect of the present invention disclosed is a method for minimizing the stains on the fabrics caused by antiperspirant composition comprising the step of (a) applying a composition according to the first aspect of the present invention on to a body part, which comes in contact with the fabric when worn by an individual, preferably the axilla followed by (b) washing, (c) preferably rinsing and (d) optionally drying the fabric.

As used herein by the term “body oil” refers to a clear, hypotonic biofluid produced by eccrine and apocrine glands located in the epidermis. Sweat is slightly acidic (pH range 4-6) and is composed mainly of water (99%), containing electrolytes (e.g., sodium, chloride, and potassium ions), urea, pyruvate, and lactate, but also proteins, peptides, amines, amino acids, and metal ions. Present in smaller concentrations are antigens, antibodies, and a variety of substances that are foreign to the body (“xenobiotics”) such as drugs, cosmetics, and ethanol. These substances are stored in sweat glands, secreted into the sweat, and transported to the epidermis with partial reabsorption of sodium and chloride during transportation.

As used herein, the term “fabric” applies to any type of cloth or textile comprising natural and/or synthetic materials. The fabrics useful in the present invention include, but are not limited to, woven, non-woven and knitted fabrics of natural fibers, synthetic fibers and mixtures of natural and synthetic fibers. Suitable fibers include, but are not limited to, fibers of cotton, linen, silk, wool, nylon, polyester, acrylic, rayon, acetate and mixtures thereof.

Herein, preferred features of the invention apply equally to all aspects of the invention. Herein, the word “comprising” is intended to mean “including” but not necessarily “consisting of” or “composed of.” In other words, the listed steps or options need not be exhaustive. Herein, all percentages are percentages by weight, unless otherwise indicated. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Herein, numerical ranges expressed in the format “from x to y” include x and y. When for a specific feature multiple preferred ranges are described in the format “from x to y”, it is understood that all ranges combining the different endpoints are also contemplated.

### DETAILED DESCRIPTION OF THE INVENTION

According to a first aspect of the present invention disclosed is an anhydrous aerosol antiperspirant composition having a metal based antiperspirant active, nonionic surfactant, and cationic surfactant.

### Nonionic Surfactant

According to the first aspect of the present invention disclosed an anhydrous aerosol antiperspirant composition which includes a non-ionic surfactant having a HLB value less than 12. More preferably the nonionic surfactant has an HLB value of less than 11, still more preferably less than 10.

As used herein, the term “HLB” refers to the “hydrophilic-lipophilic balance” of a molecule, such as a surfactant. The HLB number increases with increasing hydrophilicity. The HLB system is a semi-empirical method to predict what type of surfactant properties a molecular structure will provide. The HLB system is based on the concept that some molecules have hydrophilic groups, other molecules have lipophilic groups, and some have both. The HLB of a surfactant can be calculated according to Griffin W C: “Classification of Surface-Active Agents by ‘HLB,’” Journal of the Society of Cosmetic Chemists 1 (1949): 311; and Griffin W C: “Calculation of HLB Values of Non-Ionic Surfactants,” Journal of the Society of Cosmetic Chemists 5 (1954): 259. The HLB of a mixture of surfactants may be calculated by multiplying the proportion of each surfactant in the mixture by its HLB value and adding up the resulting values, as is known in the art.

HLB is the hydrophilic lipophilic balance which is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20.

Non-limiting examples of nonionic surfactants (along with their HLB values indicated inside the brackets) which have a HLB value of less than 12, preferably less than 11 includes but is not limited to Glycol Distearate (1), Sorbitan Trioleate (1.8), Propylene Glycol Isostearate (2.5), Glycol Stearate (2.9), Sorbitan Sesquioleate (3.7), Glyceryl Stearate (3.8), Lecithin (4), Sorbitan Oleate (4.3), Sorbitan Monostearate NF (4.7), Sorbitan Stearate (4.7), Sorbitan Isostearate (4.7), Steareth-2 (4.9), Oleth-2 (4.9), Glyceryl Laurate (5.2), Ceteth-2 (5.3), PEG-30 Dipolyhydroxystearate (5.5), Glyceryl Stearate SE (5.8), Sorbitan Stearate (and) Sucrose Cocoate (6), PEG-4 Dilaurate (6), Methyl Glucose Sesquistearate (6.6), PEG-8 Dioleate (8), Sorbitan Laurate (8.6), PEG-40 Sorbitan Peroleate (9), Laureth-4 (9.7), PEG-7 Glyceryl Cocoate (10), PEG-20 Almond Glycerides (10), PEG-25 Hydrogenated Castor Oil (10.8).

Preferably the non-ionic surfactant is selected from the group consisting of polyoxyethylene sorbitan alkyl esters, fatty alcohol ethoxylates, sorbitan monoester, alkyl polyglucosides and C₈ to C₁₆ fatty alcohol glycoside. More preferably where the non-ionic surfactant has an HLB of less than 11.0.

Preferably the non-ionic surfactant is selected from the group consisting of polyoxyethylene sorbitan alkyl esters (sold as Tween surfactants), fatty alcohol ethoxylates (sold as Brij surfactants), sorbitan monoester (sold as Span surfactants), alkyl polyglucosides and C₈ to C₁₆ fatty alcohol glycoside. More preferably where the non-ionic surfactant has an HLB of less than 11.0.

Still more preferably the nonionic surfactant for use in the present invention is selected from polyethylene glycol dodecyl ether (sold as Brij L4), Steareth-2 (sold as Brij S2), tri (oxyethylene) dodecyl ether (sold as EO3) or a combination of EO3 and hepta (oxyethylene) dodecyl ether (sold as EO7).

The antiperspirant composition according to the present invention, in addition to the nonionic surfactant already disclosed above may include additional nonionic surfactant with HLB value of less than 12, preferably the additional nonionic surfactant is a nonionic silicone surfactant. Still more preferably the nonionic silicone surfactant is a dimethicone based silicone surfactant. Preferably a dimethicone based silicone surfactant having an HLB value of less than 12 is a methyl substituted polyorganosiloxanes.

Preferred additional nonionic silicone surfactant includes silicone copolyol where polyethylene glycol and polypropylene glycol are copolymerized with dimethicone either individually or together. These combinations may be either graft, block, or end terminated. Graft polymers are those where the EO or PO chains are attached at various points along the main silicone backbone. Block or end terminated polymers have the EO or PO chains at the end of the silicone backbone. Preferably the chains may be either mixed or homopolymers. The preferred number of repeating units is below 20 and most preferred below 18.

Preferably the additional nonionic silicone surfactant is an alkyl dimethicone copolyol corresponding to the formula (I) below:

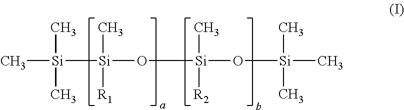

wherein;
R₁ denotes a linear or branched, C₁-C₂₀, preferably C₁-C₁₆, alkyl group
R₂ denotes the group: CₙH2ₙ—(—OC₂H₄—)ₓ—(—OC₃H₆—)_{y}—O—R₃;
R₃ denotes a hydrogen atom or linear or branched alkyl radical containing from 1 to 12 carbon atoms;
a is an integer ranging from 1 to 500
b denotes an integer ranging from 1 to 500
n is integer ranging from 2 to 12, and preferably from 2 to 5
x denotes an integer ranging from 1 to 50
y denotes an integer ranging from 0 to 50
with the proviso that, when y is other than zero, the ratio x/y is greater than 1, and preferably ranges from 2 to 11.

Among the alkyl dimethicone copolyols of formula (I) the preferred includes cetyl PEG/PPG-10/1 dimethicone, and more particularly the cetyl PEG/PPG-10/1 dimethicone and dimethicone (INCI name) mixture, such as the product sold under the trade name Abil EM90 by the company Evonik Industries.

Preferred additional nonionic silicone surfactant may have the general formula (II)

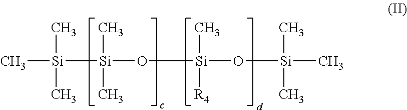

wherein;
R₄ denotes the group CₘH₂ₘ—(—OC₂H₄—)ₛ—(—OC₃H₆—)ₜ—O—R₅;
R₅ denotes a hydrogen atom or linear or branched alkyl radical containing from 1 to 12 carbon atoms;
c is an integer ranging from 1 to 500
d denotes an integer ranging from 1 to 500
m is integer ranging from 2 to 12, and preferably from 2 to 5
s denotes an integer ranging from 1 to 50
t denotes an integer ranging from 0 to 50
with the proviso that the sum of s+t is greater than or equal to 1.

Among the dimethicone copolyols of formula (II) the preferred includes PEG-18/PPG-18 dimethicone, and more particularly the cyclopentasiloxane (and) PEG-18/PPG-18 dimethicone (INCI name) mixture, such as the product sold by the Dow Chemicals under the trade name Dowsil 5225 C.

The dimethicone based nonionic silicone surfactant may also include those under the general formula (III)

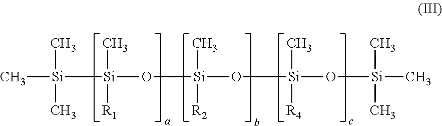

R₁ denotes a linear or branched, C₁-C₂₀, preferably C₁-C₁₆, alkyl group
R₂ denotes the group: CₙH2ₙ—(—OC₂H₄—)ₓ—(—OC₃H₆—)_{y}—O—R₃;
R₃ denotes a hydrogen atom or linear or branched alkyl radical containing from 1 to 12 carbon atoms;
R₄ denotes CₘH2ₘ—R₅
R₅ denotes hydrogen atom or Si—(O—R₆)₃
R₆ denotes a linear or branched, C₁-C₁₀, preferably C₁-C₅, alkyl group
a is an integer ranging from 1 to 500
b denotes an integer ranging from 1 to 500
c denotes an integer ranging from 0 to 500, b+c≥1
n is integer ranging from 2 to 12, and preferably from 2 to 5
m is an integer ranging from 2 to 4
x denotes an integer ranging from 1 to 50
y denotes an integer ranging from 0 to 50
with the proviso that, when y is other than zero, the ratio x/y is greater than 1, and preferably ranges from 2 to 11.

More preferably the nonionic silicone surfactant is selected from non-limiting examples which includes Lauryl PEG-8-Dimethicone sold by Siltech Corporation under the tradename Silube J208-412 (HLB value 8 to 10), Lauryl-PEG-10-tris(trimethylsiloxy) silylethyldimethicone sold by Dow chemicals under the trade name Dowsil ES 5300 (HLB value 3), PEG/PPG-18/18 dimethicone (and) cyclopentasiloxane, bis-PEG/PPG-14/14Dimethicone (and) dimethicone, and more particularly the bis-PEG-14/PPG-14 dimethicone (and) cyclopentasiloxane (INCI name) mixture, such as the product sold by the Evonik under the trade name Abil EM 97, (HLB value 5), PEG/PPG-18/18 Dimethicone (and) cyclopentasiloxane, commercially available in a mixture with cyclopentasiloxane (tradename Dowsil 5225C), cetyl PEG/PPG-10/1 Dimethicone (tradename Abil® EM 90, with HLB value 5), all commercially available from the respective companies as mentioned.

The nonionic silicone surfactant may preferably be a PEG-x-Dimethicone with x ranging from 1 to 18, still preferably from 1 to 10.

The composition according to the present invention may include other silicone-based compound for example cyclopentasiloxane (not a nonionic surfactant in accordance with the present invention), however when present the amount of the other silicone-based compound present in the antiperspirant composition is preferably lower than the amount of the metal based antiperspirant active. More preferably the weight ratio of the antiperspirant active to other silicone-based compound present in the composition is 2:1 or higher.

The nonionic surfactant is present in an amount ranging from 0.5 wt. % to 6 wt. % by weight of the composition, more preferably the nonionic surfactant is present in an amount ranging from 0.5 wt. % to 5 wt. %. Preferably the antiperspirant composition comprises at least 0.8 wt. %, still preferably at least 1 wt. %, still preferably at least 2 wt. %, most preferably at least 2.5 wt. % of the nonionic surfactant, but typically not more than 4.75 wt. %, still preferably not more than 4.5 wt. %, most preferably not more than 3.5 wt. % of nonionic surfactant having an HLB value less than 12 based on the weight of the antiperspirant composition according to the present invention.

### Antiperspirant Active

The anti-perspirant composition according to this invention includes a metal based antiperspirant active. This may be selected from an aluminium, zirconium, or mixed aluminium/zirconium salts, preferably, aluminium chlorohydrate, aluminum-zirconium tetrachlorohydrex glycine complex, aluminum-zirconium octachlorohydrex glycine complex, aluminum-zirconium pentachlorohydrate, aluminum sesquichlorohydrate (ASCH) or mixtures thereof.

Antiperspirant actives for use herein are selected from aluminium, zirconium, and mixed aluminium/zirconium salts, including both inorganic salts, salts with organic anions and complexes. Particularly preferred astringent salts are halohydrate salts, and especially chlorohydrate salts, optionally activated. For aerosol compositions, the antiperspirant active is preferably free from zirconium.

Aluminium halohydrates are usually defined by the general formula Al₂(OH)ₓQ_{y}·WH₂O in which Q represents a halogen selected from the group consisting of chlorine, bromine iodine or mixtures thereof, x is variable from 2 to 5 and x+y=6 while wH2O represents a variable amount of hydration. Especially effective aluminium halohydrate salts, known as activated aluminium chlorohydrates, are described in EP-A-6739 (Unilever N V et al), the contents of which specification is incorporated herein by reference.

The term aluminium chlorohydrate herein encompasses materials with specified figures for x and y, such as aluminium sesquichlorohydrate and materials in which the chlorohydrate is present as a complex. It will be recognized that alternative names are sometimes used to indicate the presence of hydroxyl substitution, including aluminium hydroxy chloride, aluminium oxychloride, or basic aluminium chloride.

Zirconium astringent salts for employment herein as the metal based antiperspirant active can usually be represented by the empirical general formula: ZrO(OH)_{2n-nz}B_{z}·WH₂O in which z is a variable in the range of from 0.9 to 2.0 so that the value 2_{n-nz} is zero or positive, n is the valency of B, and B is selected from the group consisting of chloride, other halide, sulphamate, sulphate or mixtures thereof. Possible hydration to a variable extent is represented by wH₂O. Preferably, B represents chloride. Preferably, the variable z lies in the range from 1.5 to 1.87. In practice, such zirconium salts are commonly not employed by themselves, but as a component of a combined aluminium and zirconium-based antiperspirant active.

The above aluminium and zirconium salts may have coordinated and/or bound water in various quantities and/or may be present as polymeric species, mixtures, or complexes. In particular, zirconium hydroxy salts often represent a range of salts having various amounts of the hydroxy group. Zirconium aluminium chlorohydrate may be particularly preferred.

Antiperspirant complexes based on the above-mentioned astringent aluminium and/or zirconium salts can be employed as metal based antiperspirant active. The complex often employs a compound with a carboxylate group, and advantageously this is an amino acid. Examples of suitable amino acids include dl-tryptophan, dl-β-phenylalanine, dl-valine, dl-methionine, and β-alanine, and preferably glycine which has the formula CH₂(NH₂)COOH. Certain of those Al/Zr complexes are commonly called ZAG in the literature. ZAG actives generally contain aluminium, zirconium, and chloride with an Al/Zr ratio in a range from 2 to 10, especially 2 to 6, an Al/Cl ratio from 2.1 to 0.9 and a variable amount of glycine. Actives of this preferred type are available from Westwood, from Summit and from Reheis. Alternatively, the complex can be pre-formed with a polyhydric aliphatic alcohol, such as propylene glycol or glycerol. A complex with a chlorohydrate is commonly referred to as a chlorhydrex.

Mixtures of two or more astringent salts can be employed, but, however, it is particularly preferred to employ astringent salts that are free from zirconium, such as aluminium chlorohydrates and so-called activated aluminium chlorohydrates.

According to an especially preferred aspect of the present invention the metal based antiperspirant active is aluminium chlorohydrate, aluminum sesquichlorohydrate or mixtures thereof.

Preferably the metal based antiperspirant active is in particulate form and is incorporated into the antiperspirant composition in the form of a suspension. The anhydrous aerosol composition preferably includes metal based antiperspirant active which has a weight average particle size ranging from 10 micrometers to 125 micrometers.

The metal based antiperspirant active preferably is included in an amount ranging from 1 wt. % to 50 wt. %, more preferably 2 wt. % to 25 wt. %, most preferably 2 wt. % to 15 wt. % by weight of the composition. Also preferred are those where the metal based antiperspirant active is present in an amount ranging from 0.5 wt. % to 10 wt. % by weight of the composition.

The metal based antiperspirant active is more preferably present in an amount ranging from 2 wt. % to 30 wt. % in the antiperspirant composition. Preferably the antiperspirant composition comprises at least 3 wt. %, still preferably at least 5 wt. %, still preferably at least 6 wt. %, most preferably at least 10 wt. % of the metal based antiperspirant active, but typically not more than 25 wt. %, still preferably not more than 20 wt. %, still further preferably not more than 18 wt. %, still more preferably not more than 16 wt. % and most preferably not more than 15 wt. %, of a metal based antiperspirant active based on the weight of the antiperspirant composition.

More particularly antiperspirant composition includes antiperspirant active in an amount ranging from 26 wt. % to 50 wt. % it was found that the yellowish stains to be particularly evident. These compositions also showed white mark formation on dark colored fabrics and the problem of fabric stiffness was also seen. It is surprisingly found that disclosed antiperspirant composition provides for minimizing the yellowish stains, white marks, and fabric stiffness when the antiperspirant composition includes 0.5 wt. % to 6 wt. % nonionic surfactant having an HLB less than 12, more particularly when the composition includes a combination of a nonionic surfactant having an HLB less than 12 and 0.5 wt. % to 6 wt. % cationic surfactant according to the invention.

Still more particularly, in concentrated anhydrous aerosol antiperspirant composition including high levels of antiperspirant active ranging from 8 wt. % to 25 wt. %, still more preferably from 10 wt. % to 20 wt. % it was found that the yellowish stain is particularly evident. These compositions also showed white mark formation on dark colored fabrics and the problem of fabric stiffness was also seen. It is surprisingly found that the method of the present invention provides for minimizing the yellowish stains, white marks and fabric stiffness when the antiperspirant composition includes a nonionic surfactant having an HLB less than 12, more particularly when the composition includes a combination of a 0.5 wt. % to 6 wt. % nonionic surfactant having an HLB less than 12 and a cationic surfactant according to the invention. Preferably the concentrated aerosol antiperspirant composition includes from 50 wt. % to 75 wt. % propellant gas and from 8 wt. % 25 wt. %, still preferably from 10 wt. % to 22 wt. % carrier oil.

### Cationic Surfactant

According to the first aspect of the present invention disclosed antiperspirant composition includes a cationic surfactant.

Cationic surfactant preferably has a quaternary ammonium group. Preferably the cationic surfactant is a quaternary ammonium compound which includes the organic ammonium compound with quaternary nitrogen atoms. They are produced by the conversion of tertiary amines with alkylation agents, such as, e.g., methyl chloride, benzyl chloride, dimethyl sulfate, dodecyl bromide, as well as ethylene oxide. Depending on the tertiary amine used, a distinction is made into three groups:

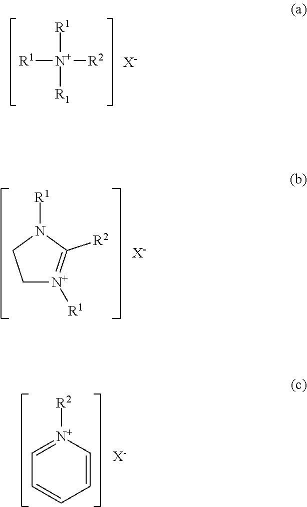

wherein R¹═CH₃, R²═C₈ to C₁₈, X=Halogen
a) Linear alkyl ammonium compounds, b) imidazolinium compounds, c) pyridinium compounds.

Linear alkyl ammonium compounds are preferred according to the invention. Preferred quaternary ammonium compounds are ammonium halides, in particular chlorides and bromides.

Formulations according to the invention advantageously contain at least one compound having the formula (IV)

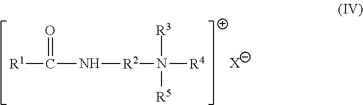

R¹ represents a saturated or unsaturated, linear, branched, or cyclic alkyl or aryl group having 8 to 24 carbon atoms, more preferably R¹ represents a saturated, linear C₁₀ to C₁₈ alkyl group.

R² represents a saturated or unsaturated, linear, branched, or cyclic substituted or non-substituted alkylene group having 1 to 10 carbon atoms and 0 to 5 hydroxy groups, more preferably the R² represents the group CH₂CH₂CH₂—. R³, R⁴, R⁵ independently of one another represent hydrogen, a C₁ to C₄ alkyl group, a C₅ or C₆ cycloalkyl group, an aryl group or a C₂ to C₄ hydroxy alkyl group, and X represents a monovalent anion, more preferably radicals R³, R⁴,

R⁵ each represent alkyl group, for example but not limited to methyl, ethyl, propyl, isopropyl and butyl groups. In particular methyl groups are preferred.

Preferably compounds according to formula (IV) are those in which the monovalent anion X represents halide, for example chloride or bromide or one of the two groups CH₃—O—SO₃ (methosulfate) or CH₃CH₂—O—SO₃ (ethosulfate).

More preferably the cationic surfactants include compound according to the Formula (IV) includes palmitamidopropyltrimonium chloride and commercially available under the trade name Varisoft®PATC (Ex. Evonik), behenamidopropyl ethyldimonium ethosulfate, such as is commercially available for example under the trade name Mackernium BAPDES from Mcintyre, stearamidopropyl trimonium methosulfate, as is commercially available for example under the trade name Catigene SA-70 from Stepan Company and/or undecylamidopropyltrimonium methosulfate, such as is available for example under the trade name Rewocid UTM 185 from Evonik. Varisoft®PATC is composed of 60% palmitamidopropyltrimonium chloride in 40% propylene glycol as solvent. Alternatively, a mixture of palmitamidopropyltrimonium chloride and a fatty alcohol (cetearyl alcohol) can be used, trade name Tego Care CE 40.

Preferably the cationic surfactant has a structure represented by

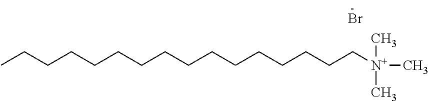 [SMILES: Br ; CCCCCCCCCCCCCCCC[N+](C)(C)C]

The cationic surfactant is included in an amount ranging from 0.1 wt. % to 4 wt. % by weight of the composition, more preferably in an amount ranging from 0.5 wt. % to 4 wt. % by weight of the composition. Preferably the antiperspirant composition comprises at least 0.3 wt. %, 0.5 wt. %, still preferably at least 0.8 wt. %, still preferably at least 1 wt. %, most preferably at least 1.5 wt. % of the cationic surfactant, but typically not more than 4 wt. %, more preferably not more than 3.75 wt. %, still preferably not more than 3.6 wt. %, not more than 3.5 wt. %, most preferably not more than 3 wt. % of cationic surfactant based on the weight of the antiperspirant composition.

Preferably the weight ratio of metal based antiperspirant active to the cationic surfactant is preferably 20:1 to 30:1, more preferably 10:1 to 15:1, most preferably 5:1 to 8:1.

Preferably the weight ratio of the cationic surfactant to the nonionic surfactant is preferably ranging from 1:1 to 1:10, more preferably 1:1 to 1:5, still more preferably 1:1 to 3:1, also preferably from 1:1.5:1:10, 1:1.5:1:5, and most preferably from 1:1 to 1:3 or 1:1.5 to 1:3. It was surprising found that when the weight. % of the cationic surfactant is lower than the weight % of the nonionic surfactant in the anhydrous aerosol antiperspirant composition it provides for improved stain removal performance.

### Emulsifier

According to the first aspect of the present invention disclosed antiperspirant composition preferably includes an emulsifier. By the term emulsifier it is meant that the ingredient ensures that two immiscible liquids (for example oil and water) can mix to form an emulsion.

Nonionic emulsifiers include fatty alcohols (e.g., cetyl or stearyl alcohol), partial fatty acid esters of polyhydric alcohols with saturated fatty acids (e.g., glycerol monostearate), partial fatty acid esters of polyhydric alcohols with unsaturated fatty acids (eg., glycerol monooleate, pentaerythritol monooleate), also polyoxyethylene esters of fatty acids (eg., polyoxyethylene stearate), polymerization products of ethylene oxide and propylene oxide with fatty alcohols (fatty alcohol polyglycol ether) or fatty acids (fatty acid ethoxylates).

It is preferred that the anhydrous aerosol antiperspirant composition according to the present invention includes minimal or no nonionic emulsifier with an HLB of more than 12.

Preferably the anhydrous aerosol antiperspirant composition according to the present invention includes less than 10 wt. % nonionic emulsifier, more preferably less than 5 wt. % nonionic emulsifier, still preferably less than 3 wt. %, still more preferably less than 1 wt. %, and most preferably the anhydrous aerosol antiperspirant composition has 0 wt. % nonionic emulsifier. Non-limiting examples of nonionic emulsifier includes fatty alcohols such as stearyl alcohol, octyldodecanol, and cetyl alcohol.

The above-mentioned non-ionic emulsifier do not count among the nonionic surfactants according to the invention.

### Unsaturated Oil

According to the first aspect of the present invention in disclosed antiperspirant composition preferably includes an unsaturated oil. The anti-perspirant composition according to the first aspect of the present invention includes an unsaturated oil having an iodine value of at least 70, preferably at least 80, still preferably at least 90, and also more preferably at least 100.

As used herein, “iodine value” is the number of grams of iodine that an unsaturated compound or blend will absorb in a given time under arbitrary conditions. A low iodine value implies a high level of saturation, and vice-versa. The iodine value can be determined by the WIJ'S method of the American Oil Chemists Society (A.O.C.S. Cd1-25).

The unsaturated oil are preferably natural oils, preferably a glyceride oil derived from one or more unsaturated fatty acids. In many instances, the oils comprise one or more triglycerides. The fatty acid residues in the oil can comprise, commonly from one to three olefinic unsaturated bonds and often one or two. Whilst in many instances the olefinic bonds adopt the trans configuration, in a number of desirable products the bond or bonds adopt the cis configuration. If two or three olefinic unsaturated bonds are present, they can be conjugated. The fatty acid can also be substituted by a hydroxyl group.

The unsaturated natural oils employable herein desirably comprise one or more triglycerides of oleic acid, linoleic acid, linolenic acid or ricinoleic acid. Various isomers of such acids often have common names, including linolenelaidic acid, trans 7-octadecenoic acid, parinaric acid, pinolenic acid punicic acid, petroselenic acid and stearidonic acid. It is especially desirable to employ glycerides derived from oleic acid, linoleic acid or petroselenic acid, or a mixture containing one or more of them.

Natural unsaturated oils containing one or more of such triglycerides include coriander seed oil for derivatives of petroselinic acid, impatiens balsimina seed oil, parinarium laurinarium kernel fat or sabastiana brasilinensis seed oil for derivatives of cis-parinaric acid, dehydrated castor seed oil, for derivatives of conjugated linoleic acids, borage seed oil and evening primrose oil for derivatives of linoleic and linolenic acids, aquilegia vulgaris oil for columbinic acid and sunflower oil, olive oil or safflower oil for derivatives of oleic acid, often together with linoleic acids. Other suitable oils are obtainable from hemp, which can be processed to derive stearadonic acid derivatives and maize corn oil. An especially convenient natural oil by virtue of its characteristics and availability comprises sunflower oil, ranging from those rich in oleic acid glycerides to those rich in linoleic acid glycerides, rich indicating that its content is higher than that of the other named acid.

Preferably the unsaturated oil is a natural oil which is still preferably selected from at least one of coriander seed oil, borage seed oil, evening primrose oil, maize corn oil, sunflower oil, safflower oil, algal oil, or mixtures thereof. More preferred natural oils are sunflower oil, algal oil, preferably sunflower oil. The unsaturated oil is preferably included in an amount ranging from 0.05 wt. % to 10 wt. %, more preferably 1 wt. % to 5 wt. % % by weight of the composition.

Preferably the unsaturated oil is present in an amount ranging from 0.05 wt. % to 10 wt. % in the antiperspirant composition. Preferably the antiperspirant composition comprises at least 0.08 wt. %, still preferably at least 0.1 wt. %, still preferably at least 0.3 wt. %, most preferably at least 0.5 wt. % of the metal based antiperspirant active, but typically not more than 8 wt. %, still preferably not more than 6 wt. %, still further preferably not more than 5 wt. %, still more preferably not more than 4 wt. % and most preferably not more than 3 wt. %, of an unsaturated oil based on the weight of the antiperspirant composition.

### Antiperspirant Composition

By “An Antiperspirant Composition” as used herein, is meant to include a composition for topical application to the skin of mammals, especially humans. Such a composition is preferably of the leave-on type. By a leave-on composition is meant a composition that is applied to the desired skin surface and left on for a period of time (say from one minute to 24 hours) after which it may be wiped or rinsed off with water, usually during the regular course of personal washing. The composition may also be formulated into a product which is applied to a human body for improving the appearance, cleansing, odor control or general aesthetics. The composition of the present invention can be in the form of a liquid, foam, spray, aerosol, gel or stick form. “Skin” as used herein is meant to include skin on any part of the body (e.g., neck, chest, back, arms, underarms, hands, legs, buttocks, and scalp) especially the underarms. Alternatively, they may be delivered through a roll-on device or by using a propellant containing aerosol can. It is especially useful for delivering low pH compositions to the axilla of an individual for anti-perspirancy benefits. “Skin” as used herein is meant to include skin on any part of the body (e.g., neck, chest, back, arms, underarms, hands, legs, buttocks, and scalp) especially the underarms.

The composition of the invention is substantially anhydrous. By “anhydrous” is meant that the composition comprises no added water. However, the composition may comprise some water that gets included in the composition from the ingredients formulated to form the composition. The water content may also be as a result of the ingress of water from the moisture in the atmosphere which reaches an equilibrium with the components of the composition. In effect, the composition comprises less than less than 10 wt. %, preferably less than 5 wt. %, furthermore preferably less than 2 wt. %, and most preferably less than 0.5 wt. % by weight of the composition.

The composition of the present invention is an anhydrous aerosol antiperspirant composition.

Preferably the anhydrous aerosol composition includes a structurant. Nonlimiting examples of the structurant includes disteardimonium hectorite, propylene carbonate and more preferably a combination thereof. Preferably the amount of structurant present in the composition ranges from 0.01 wt. % to 4 wt. %, still preferably 0.01 wt. % to 1 wt. %, still preferably from 0.01 wt. % to 0.8 wt. %, still preferably from 0.01 wt. % to 0.7 wt. %.

### Optional Ingredients

According to the first aspect of the present invention in disclosed antiperspirant composition may optionally contain additives customary in cosmetics, for example, perfume, thickeners, deodorants, antimicrobial substances, regreasing agents, complexing and masking agents, pearlescent agents, plant extracts, vitamins, active ingredients, preservatives, bactericides, dyes, pigments that have a coloring effect, thickeners, moisturizing and/or humectant substances, fats, oils, waxes or other usual constituents of a cosmetic or dermatological formulation, such as alcohols, polyols, polymers, foam stabilizers, electrolytes, organic solvents or silicone derivatives, unless they are counter to the composition according to the invention and the use thereof.

### Moisturizer:

Moisturizers can also advantageously be used as anti-wrinkle substances for protection against aging of the skin, such as occur, e.g., in skin aging.

### Antioxidant Compound:

According to the first aspect of the present invention in disclosed antiperspirant composition may include an antioxidant compound. Suitable and conventional antioxidants which are generally included in such compositions may be added in the present invention. Such antioxidants are selected from one or more of butyl hydroxy toluene or its derivatives, pentaerythrityl tetra-di-t-butyl hydroxyhydrocinnamate (sold as Tinogard TT), or dilauryl thiodipropionate (sold as Tinogard DA), Tetramethylhydroxypiperidinol) Citrate (sold as Tinogard Q), tocopherol acetate or ascorbic acid and its derivatives. The antiperspirant composition preferably comprises 0.001 wt. % to 10 wt. %, more preferably 0.01 wt. to 5 wt. %, and most preferably 0.01 wt. to 2 wt. % antioxidant compound by weight of the composition.

According to the first aspect of the present invention in disclosed antiperspirant composition preferably includes one or more optional ingredients selected from polyhydric alcohol.

According to the first aspect of the present invention in disclosed antiperspirant composition preferably includes a polyhydric alcohol. Polyhydric alcohol is also referred to in short as polyol. A polyhydric alcohol as per the present invention is a compound having two or more hydroxyl groups. Suitable class of polyhydric alcohols that may be included in the composition of the invention are monomeric polyols, polyalkylene glycols or sugars. Preferred monomeric polyols are glycol; alkylene glycol e.g., propylene glycol; glycerol; or xylitol, more preferably propylene glycol.

Suitable polyalkylene glycols are polyethylene glycol or polypropylene glycol. Sugars for inclusion in the invention could be monomeric, dimeric, trimeric or of the polymeric form. Preferred sugars include glucose, fructose, mannose, sucrose, threitol, erythritol, sorbitol, mannitol, galactitol, adonitol, dextran, or cyclodextrin. Of these the more preferred sugars are glucose, fructose, sucrose, sorbitol, mannitol, adonitol, dextran, or cyclodextrin.

Other components commonly included in conventional antiperspirant compositions may also be incorporated in the composition of the present invention. Such components include skin care agents such as emollients, humectants, and skin barrier promoters; skin appearance modifiers such as skin lightening agents and skin smoothing agents; antimicrobial agents, in particular organic anti-microbial agents, and preservatives.

Preferably the antiperspirant composition comprises antimicrobial agents. Preferably the antiperspirant composition comprises perfume.

The composition of the invention is preferably delivered as a composition in aerosol form, spray form or in stick form or roll on. Preferably the antiperspirant composition is in an anhydrous composition.

### Stick or Soft Solid Composition

Many different materials have been proposed as gellant for a continuous oil phase, including waxes, small molecule gelling agents and polymers. The sticks preferably include suspension sticks and opaque emulsion sticks. They each have their advantages and of them, one of the most popular class of gellant has comprised waxes, partly at least due to their ready availability and ease of processing, including in particular linear fatty alcohol wax gellants. A gelled antiperspirant composition is applied topically to skin by wiping it across and in contact with the skin, thereby depositing on the skin a thin film. The nature of the film depends to a significant extent on the gellant that is employed. Although wax fatty alcohols have been employed as gellant for many years, and are effective for the purpose of gelling, the resultant product is rather ineffective at improving the visual appearance of skin, and in particular underarm skin, to which the composition has been applied. This problem has been solved by including ameliorating materials for example, di or polyhydric humectants and/or a triglyceride oil.

### Roll-on Compositions

Liquid compositions that are applicable from a roll-on broadly speaking can be divided into two classes, namely those in which an antiperspirant active is suspended in a hydrophobic carrier, such as a volatile silicone and those in which the antiperspirant active is dissolved in a carrier liquid. The latter has proven to be more popular. There are mainly two sorts of dissolving carrier liquid, namely carriers that are predominantly alcoholic, which is to say the greater part of the dissolving carrier fluid comprises ethanol and the second class in which the carrier liquid is mainly water. The former was very popular because ethanol is a mild bactericide in its own right.

### Aerosol Composition

The antiperspirant composition according to the present invention is delivered through an aerosol composition. The aerosol composition preferably comprises a propellant in addition to the other ingredients described hereinabove. Commonly, the propellant is employed in a weight ratio to the base formulation of from 95:5 to 5:95. Depending on the propellant, in such aerosol compositions the ratio of propellant to base formulation is normally at least 20:80, generally at least 30:70, particularly at least 40:60, and in many formulations, the weight ratio is from 90:10 to 50:50. A ratio range of from 70:30 to 90:10 is sometimes preferred.

Propellants herein generally are one of three classes; i) low boiling point gasses liquifided by compression, ii) volatile ethers and iii) compressed non-oxidising gases. Class i) is conveniently a low boiling point material, typically boiling below −5° C., and often below −15° C., and in particular, alkanes and/or halogenated hydrocarbons. This class of propellant is usually liquefied at the pressure in the aerosol canister and evaporates to generate the pressure to expel the composition out of the canister. Examples of suitable alkanes include particularly propane, butane, or isobutane. The second class of propellant comprises a very volatile ether of which the most widely employed ether hitherto is dimethyl ether. This propellant can advantageously be employed at relatively low weight ratio of propellant to base formulation, for example to as low as 5:95. It can also be employed in admixture with, for example, compressible/liquefiable alkane gasses. The third class of propellant comprises compressed non-oxidizing gasses, and in particular carbon dioxide or nitrogen. Inert gases like neon are a theoretical alternative. Also preferred are hydrocarbons or halogenated hydrocarbon gases (particularly fluorinated hydrocarbons such as 1,1-difluoroethane and/or 1-trifluoro-2-fluoroethane) that have a boiling point of below 10° C. and especially those with a boiling point below 0° C.

When the composition comprises a propellant, it is delivered as an aerosol.

The composition of the present invention can comprise a wide range of other optional components. The CTFA Personal care Ingredient Handbook, Second Edition, 1992, which is incorporated by reference herein in its entirety, describes a wide variety of non-limiting personal care and pharmaceutical ingredients commonly used in the skin care industry, which are suitable for use in the compositions of the present invention. Examples includes binders, biological additives, buffering agents, colorants, thickeners, polymers, astringents, fragrance, conditioners, exfoliating agents, pH adjusters, preservatives, natural extracts, essential oils, skin sensates, skin soothing agents, and skin healing agents.

The composition of the present invention is an anhydrous aerosol antiperspirant composition.

Preferably the anhydrous aerosol composition includes a structurant. Nonlimiting examples of the structurant includes disteardimonium hectorite, propylene carbonate and more preferably a combination thereof. Preferably the amount of structurant present in the composition ranges from 0.01 wt. % to 4 wt. %, still preferably 0.01 wt. % to 1 wt. %, still preferably from 0.01 wt. % to 0.8 wt. %, still preferably from 0.01 wt. % to 0.7 wt. %.

### Applying the Antiperspirant Composition

The antiperspirant composition can preferably be applied cosmetically and topically to the skin, broadly speaking, by one of two methods. Some consumers prefer one method and some others, the other method. In one method, sometimes called a contact method, a composition is rubbed across the surface of the skin, depositing a fraction of the composition as it passes. In the second method, sometimes called the non-contact method, the composition is sprayed from a dispenser held proximate to the skin. Often in an area of about 10 to 20 cm². The spray can be developed by mechanical means of generating pressure on the contents of the dispenser, such as a pump or a squeezable sidewall or by internally generated pressure arising from a fraction of a liquefied propellant volatilizing, the dispenser commonly being called an aerosol.

Preferably the anhydrous aerosol antiperspirant composition according to the present invention comprises the metal based antiperspirant active as a suspension, preferably the suspension also includes a structurant. The cationic surfactant and the nonionic surfactant is uniformly dispersed in the pressurized propellant which is preferably in the form of a monophasic liquid. The composition according to the present invention is preferably mixed by shaking the container to redisperse the suspension comprising metal based antiperspirant active, uniformly in the composition before use.

There are broadly speaking two classes of contact compositions, one of which is liquid and usually applied using a roll-on dispenser or possibly absorbed into or onto a wipe, and in the second of which the antiperspirant active is distributed within a carrier liquid that forms a continuous phase that has been gelled. In one variation, the carrier fluid comprises a solvent for the antiperspirant and in a second variation, the antiperspirant remains a particulate solid that is suspended in an oil, usually a blend of oils.

The composition is preferably applied on the axilla. The method is also preferably non-therapeutic or for cosmetic application.

Fabric staining means stains particularly in the underarm region. These are stains that remain in clothing after wearing and/or after washing and can become more intensive with increasing age of the fabric and are produced when the antiperspirant composition or constituents thereof reach the textiles during sweating together with the armpit secretions. Part of these deposits are washed out during washing, another part remains on the textile as residue.

Fabrics stains also includes the white marks which are typically produced by the transfer of antiperspirant active particles or other constituents in the antiperspirant composition from the skin to the fabric and further aggravated due to residue build-up of the antiperspirant ingredients on multiple wash wear. White spots or marks formed particularly on the dark coloured, or black fabrics are consumer perceivable. The antiperspirant composition according to the present invention provides for reducing particulate build up on the fabrics on repeated wash wear, thus reducing the white marks on dark coloured/black fabrics.

In addition to the stains the interaction between the antiperspirant composition with the body oil and deposition on the fabrics may cause stiffness of the fabrics. The antiperspirant composition according to the present invention including specific nonionic surfactant, preferably along with the cationic surfactant provides for reducing stiffness of fabrics.

The stains are further prominent when the composition includes unsaturated oil. Preferably the composition according to the first aspect of the present invention provides to reduce or prevent the formation of stains that are produced by the presence of antiperspirant active and caused by the interaction of the antiperspirant composition with the body oils or the oils present in the antiperspirant composition. The presence of nonionic surfactant having an HLB value of less than 11 in the antiperspirant composition along with a cationic surfactant improves the ability for them to washed out.

Preferably the composition according to the first aspect of the present invention provides for improving the washability of the stains formed on the fabric at least in part by the antiperspirant composition. Washability refers to the ability to clean without fading or damaging the fabric.

Preferably the composition according to the first aspect of the present invention, provides for minimizing of staining or yellow discoloration on a fabric in contact with the antiperspirant composition which is noticeable after laundering the fabric. More preferably from the first wash. This is seen through a lower b value of the fabric after washing the fabric.

### EXAMPLES

The invention will now be illustrated with the help of the following non-limiting examples.

Anhydrous antiperspirant aerosol composition as shown in Table 1 below were prepared:

### Example 1: Effect of Presence of a Non-Ionic Surfactant in Accordance to the Present Invention

A composition according to the present invention having a metal based antiperspirant active was studied for the effect of inclusion of specific levels of non-ionic surfactant and cationic surfactant as in Table 1, and the cleaning efficacy was measured as given in the protocol below.

### Protocol Used for the Evaluation:

Studies were carried out, to evaluate the performance of the compositions on stains formed by the interaction of body oil and antiperspirant active.

Stain removal measurement: In the study, a 12×12 cm² fabric swatch was taken. In this study 0.3 g of APA base was premixed with 50 mg of model sebum and applied on a circular area corresponding to 33 cm². 0.5 mL of ionic sweat was spread uniformly over the fabric swatch. Swatches were incubated at 45° C. for 12 h. The swatches were washed with Persil Non-Bio liquid detergent in Front load washing machine (make: IFB SX). Study was done with 4 replicate swatches for each sample. The swatches were dried in Tumble drier (IFB Tumble drier).

Treated fabric swatches were washed in an IFB front loader washing machine, programmed for soak (30 minutes), wash and two rinses. A wash load of approximate 2±0.1 kg including polycotton fabrics and the treated swatches were placed into the washing machine. The hardness of water was maintained at 24 FH water (2:1, Ca: Mg). 36 grams of Persil Non-Bio Liquid detergent was added in the dosing chamber at a dosage level of 3 gpL. The swatches were dried using tumble dryer (IFB) at hot air mode (45° C.) for 90 minutes. The dried swatches were ironed according to the nature of the fabric as stipulated by iron box manufacturer (Black+Decker Steam Iron).

The above process of treating the swatches followed by washing the swatches was repeated 4 times and after the final cycle the chromophoric evaluation of the swatches was conducted using a Konika Minolta spectrophotometer Model-2600d. Konika Minolta spectrophotometer was used to measure the stain intensity on the swatches. L*, a*, b* were measured at three different areas of the stain treated swatches and untreated swatches. Following which the D b* were calculated subsequently using the ASTM D4265-21 method (Standard guide for Evaluating Stain removal performance in-home laundering).

The higher the D b* the higher is the yellowness, hence lower D b* values indicated better stain removal performance.

TABLE 1

| | Ex A | Ex B | Ex 1 | Ex 2 | Ex 3 | Ex 4 | Ex 5 |
|---|---|---|---|---|---|---|---|
| Ingredient | wt % | wt % | wt % | wt % | wt % | wt % | wt % |
| Cyclopentasiloxane | 2.5 | 2.5 | 2.5 | 2.5 | 2.5 | 2.5 | 2.5 |
| PPG 14 Butyl ether | 3 | 3 | 3 | 3 | 3 | 3 | 3 |
| Sunflower seed oil | 0.5 | 0.5 | 0.5 | 0.5 | 0.5 | 0.5 | 0.5 |
| BHT | 0.1 | 0.1 | 0.1 | 0.1 | 0.1 | 0.1 | 0.1 |
| Aluminium | 5.5 | 5.5 | 5.5 | 5.5 | 5.5 | 5.5 | 5.5 |
| chlorohydrate | | | | | | | |
| Disteardimonium | 0.6 | 0.6 | 0.6 | 0.6 | 0.6 | 0.6 | 0.6 |
| hectorite | | | | | | | |
| Propylene carbonate | 0.015 | 0.015 | 0.015 | 0.015 | 0.015 | 0.015 | 0.015 |
| C12-15 alkyl benzoate | 0.5 | 0.5 | 0.5 | 0.5 | 0.5 | 0.5 | 0.5 |
| Octyldodecanol | 0.12 | 0.12 | 0.12 | 0.12 | 0.12 | 0.12 | 0.12 |
| Cationic surfactant* | 0 | 2 | 0 | 2 | 2 | 6 | 10 |
| Nonionic surfactant^{&} | 0 | 0 | 2 | 2 | 6 | 2 | 2 |
| Butane, isobutane, | To 100 | To 100 | To 100 | To 100 | To 100 | To 100 | To 100 |
| propane | | | | | | | |
| Db* value | 5.35 | 5.96 | 1.89 | 1.74 | 0.56 | 2.45 | 4.92 |
| Std dev | 0.7 | 0.6 | 0.9 | 0.6 | 0.5 | 0.4 | 0.7 |

*Cetyl trimethyl ammonium bromide
^{&}Brij L4 = Polyethylene glycol dodecyl ether (INCI name) with HLB 9

The data in the table 1 shows that the composition according to the present invention (Ex 3) where the antiperspirant composition includes a non-ionic surfactant (polyethylene glycol dodecyl ether) having HLB less than 12 at claimed levels and a cationic surfactant (cetyl trimethyl ammonium bromide) at claimed levels according to the present invention has a lower Db* value as compared to a comparative compositions (E×A and E×B) where Ex A does not have a non-ionic surfactant and cationic surfactant, and Ex B has either a cationic surfactant or nonionic surfactant but not both. All of these compositions were found to have a higher Db* value as compared to the compositions according to the invention. This clearly shows that the composition according to the invention provides for minimizing the stains on clothing caused by the interaction of body oils with antiperspirant composition having a metal based antiperspirant active or those caused by the interaction of the antiperspirant active with the oils present in the aerosol antiperspirant composition which is anhydrous and is better in performance as compared to the comparative composition.

1. An anhydrous aerosol antiperspirant composition comprising:
a a metal based antiperspirant active;
b 0.1 wt. % to 4 wt. % cationic surfactant by weight of the composition; and
c 0.5 wt. % to 6 wt. % by weight of the composition nonionic surfactant having an HLB value less than 12;
wherein:
the nonionic surfactant is selected from the group consisting of polyoxyethylene sorbitan alkyl esters, fatty alcohol ethoxylates, or sorbitan monoester, alkyl polyglucosides, and C8-C16 fatty alcohol glycoside; and
the anhydrous aerosol antiperspirant composition comprises less than 10 wt. % water content by weight of the composition.

2. The composition according to claim 1, wherein the nonionic surfactant is selected from the group consisting of polyethylene glycol dodecyl ether, Steareth-2, tri(oxyethylene) dodecyl ether (EO3) and a combination of tri (oxyethylene) dodecyl ether (EO3) and hepta (oxyethylene) dodecyl ether (EO7).

3. The composition according to claim 1, further comprising a silicone nonionic surfactant.

4. The composition according to claim 1, wherein the cationic surfactant is:
(i) a Compound having the Formula (IV): 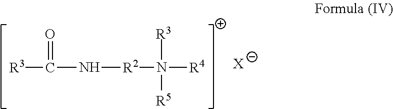
wherein:
R¹ represents a saturated or unsaturated, linear, branched, alkyl group having 8 to 24 carbon atoms;
R² represents a saturated or unsaturated, linear or branched alkylene group having 1 to 10 carbon atoms and 0 to 5 hydroxy groups;
R³, R⁴, R⁵ each independently represent hydrogen and/or a C₁ to C₄ alkyl group; and
X represents a monovalent anion;
or
(ii) a Compound represented by the structure:

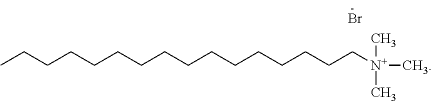 [SMILES: Br ; CCCCCCCCCCCCCCCC[N+](C)(C)C]

5. The composition according to claim 4, wherein the cationic surfactant is selected from the group consisting of palmitamidopropyltrimonium chloride. behenamidopropyl ethyldimonium ethosulfate, stearamidopropyl trimonium methosulfate, and undecylamidopropyltrimonium methosulfate.

6. The composition according to claim 1, further comprising an unsaturated oil.

7. The composition according to claim 1, wherein the water content is less than 5 wt. % by weight of the composition.

8. The composition according to claim 1, wherein the metal based antiperspirant active is selected from an aluminium, zirconium or mixed aluminium/zirconium salt, or mixtures thereof.

9. The composition according to claim 1, further comprising a fragrance oil.

10. The composition according to claim 1, wherein the metal based antiperspirant active is present an amount ranging from 1 wt. % to 50 wt. % by weight of the composition.

11. The composition according to claim 1, wherein the composition comprises 8 wt. % to 20 wt. % metal based antiperspirant active by weight of the composition.

12. The composition according to claim 1, wherein the weight ratio of the cationic surfactant to the nonionic surfactant ranges from 1:1 to 1:10.

13. The composition according to claim 1, wherein the weight ratio of metal based antiperspirant active to the cationic surfactant ranges 20:1 to 30:1.

14. The composition according to claim 1, further comprising a structurant selected from the group consisting of disteardimonium hectorite, propylene carbonate and combination thereof.

15. A method for minimizing staining on a fabric worn by an individual comprising applying the composition of claim 1 to a body part of the individual and washing the fabric.

16. A method of reducing perspiration, comprising applying the composition of claim 1 to a body part of an individual.
